(19) United States
(12) Patent Application Publication
(10) Pub. No.: US 2005/0019386 A1
(43) Pub. Date: Jan. 27, 2005

(54) Orally administered pharmaceutical preparation comprising liposomically encapsulated paclitaxel

(76) Inventors: Regina Reszka, Schwanebeck (DE); Iduna Fichtner, Berlin (DE)

Correspondence Address: NORRIS, MCLAUGHLIN & MARCUS, P.A., 875 THIRD AVE, 18TH FLOOR, NEW YORK, NY, 10022, US

(21) Appl. No.: 10/495,039

(22) PCT Filed: Nov. 6, 2002
(86) PCT No.: PCT/DE02/04120

(30) Foreign Application Priority Data
Nov. 8, 2001 (DE) 101 54 464.2

(51) Int. Cl. A61K 9/127; A61K 31/337

(52) U.S. Cl. 424/450; 514/449

(57) ABSTRACT

The invention relates to pharmaceutical preparations suitable for oral application of liposomally encapsulated Taxol, its derivatives and Taxan. Preferably, they additionally contain at least one immuno-modulator, preferably Cyclosporine, and/or at least one cytokine, preferably PEG cytokines.

The invention relates to pharmaceutical preparations suitable for oral application of liposomally encapsulated taxol, its derivatives and taxan. Fields of application of the invention are medicine and the pharmaceutical industry.

Taxol (chemically: paclitaxel) is a natural agent occurring in the bark of various species of yews (taxaceae) and can be obtained from these barks and also by chemical synthesis [J. Amer. Chem. Soc., 1110:5917-5919 (1988)]. Taxol supports the aggregation of the microtubuli from tubulindimers and stabilises the microtubuli by inhibiting their depolymerisation. In addition, there is an abnormal arrangement and bundling of microtubuli during the entire cell cycle, which leads to formation of multiple microtubular division stars during the mitosis and thus to the inhibition of the normal dynamic reorganisation of the microtubular network. As the vital cell function in the interphase and during the mitosis is decisively influenced by this, Taxol shows a distinct anti-neoplastic activity against various tumours, inter alia against implanted B16 melanoma, P388 leukaemia and against human mamma tumours.

However, the applicability of Taxol is greatly limited due to its low water-solubility. Although solution mediators such as Cremophor (poly-ethoxylated castor oil) and alcohol improve the solubility, they also lead to considerable side-effects in the application, e.g. to anaphylactic reactions. Dilution with a physiological saline solution for the application has the disadvantage that taxol does not have sufficient stability (maximum of 24 hours) in a physiological saline solution. A dose-limiting side-effect is the myelo-suppression, primarily the neutropaenia [Semin. Oncol. 19:646-662 (1992)]. Liposomes provide the possibility of including and incorporating both water-soluble and lipid-soluble substances due to their amphiphile character.

Taxol as an almost water-insoluble substance can be dissolved with high efficiency in the lipid phase by liposomes of a suitable composition, which can be used for the treatment of various kinds of tumours and localisations. In a study, taxol was tested with regard to its anti-tumour activity in a free and a liposomal form on two human glioblastoma in a nude model (12.5 mg/kg/4 days). Both forms led to a significant reduction of the growth of the tumour [In-Vivo 6 (1):23-7 (1992)].

In WO 93/18751, the encapsulation of Taxol in liposomes and the use of the products obtained for treatment of cancer diseases is described. A combination of this treatment with hyperthermia is preferred. The taxol liposomes produced manifest an improved stability. From DE 44 30 593 C2, a high-pressure homogenisation method for the production of liposomally encapsulated taxol is known, with the liposomes manifesting a high share of taxol and high stability.

Taxol can be outstandingly used as a cytostatic, although its application is limited to parenteral preparations. An effectivity of taxol in oral application has yet to be established internationally. Peroral forms of application for liposomally encapsulated taxol are also not known as yet.

It was surprisingly found that pharmaceutical preparations of liposomally encapsulated taxol can be used for oral application and manifest a good and fast, if applicable retarding effectivity in these forms of application. This effectivity was increased even further if the oral forms of application contain not only liposomally encapsulated taxol, but also at least one immuno-modulator and/or at least one cytokine. Derivatives of taxol and taxan are also effective.

As can be seen from the enclosed figure, Taxol was applied as a 50 mg bolus in an oral form of administration and the effectivity of the agent determined on the basis of the tumour mass (ovarian carcinoma (human) on a nude mouse).

Application A was used as a control, an influence on the tumour mass was not established. B and C show the effectivity of unencapsulated taxol (B) and of unencapsulated taxol in combination with Cyclosporin A after oral application, with the tumour mass hardly being reduced.

D shows the effectivity after oral application of liposomally encapsulated taxol, the tumour mass being reduced significantly.

E, which shows the effectivity of the oral application of liposomally encapsulated taxol in combination with Cyclosporin A, led to the disappearance of the tumour.

The preferred dosage for liposomally encapsulated taxol is 1×50 mg/kg body weight per day. The dosage of Cyclosporin is 5×50 mg/kg body weight per day.

The invention is implemented according to the claims.

1. A pharmaceutical preparation for oral administration, the pharmaceutical preparation comprising at least one active ingredient selected from the group consisting of paclitaxel (taxol), a derivative thereof, and taxans wherein a high share of the at least one active ingredient is encapsulated in a mixture of membrane-forming amphiphiles; in which the active ingredient was dissolved, and with addition of a watery phase, this mixture being subjected to high-pressure homogenisation or ultrasound.

2. The use of liposomally encapsulated paclitaxel (taxol), its derivatives or taxan for the production of a drug for oral administration in tumour therapy.

3. The pharmaceutical preparation of claim 1, further comprising at least one immuno-modulator, preferably Cyclosporine, and/or at least one cytokine.

4. The pharmaceutical preparation of claim 1, further comprising pharmaceutical ancillaries and additives customary in the art.

5. The pharmaceutical preparation of claim 1, wherein the liposomes manifesting encapsulated taxol with a high share of taxol comprise
a) a natural, semi-synthetic or fully synthetic amphiphile
b) a steroid
c) a charged lipid component and/or a saturated lipid component and/or an ether lipid component, and
d) a carrier fluid and, if applicable, additional ancillaries.

6. The pharmaceutical preparation of claim 5, wherein the amphiphile in a) is selected from the group consisting of a lipid, tenside, emulsifier, polyethylene glycol (PEG) and lipid PEG.

7. The pharmaceutical preparation of claim 6, wherein amphiphile has the general formula I, 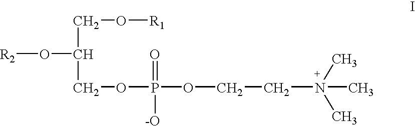
in which R₁ and R₂═C₁₀-C₂₀-alkanoyl, alkenoyl, alkyl, alkenyl.

8. The pharmaceutical preparation of claim 5, wherein the steroid is selected from the group consisting of cholesterol, diethoxy-cholesterol and sitosterol.

9. The pharmaceutical preparation of claim 5, wherein the charged lipid component of (c) is selected from the group consisting of an anion of dicethyl phosphate, of palmitine acid, of stearic acid, the anion of a phospholipid, the anion of a sphingolipid, and an anion of polyethylene glycol (PEG) is used as the charged lipid component.

10. The pharmaceutical preparation of claim 5, wherein the component (c) is selected from the group consisting of phosphatidylserine, phosphatide acid, phosphatidylglycerol and sulphatide.

11. The pharmaceutical of claim 5, wherein component (c) is phosphatidylcholine.

12. The pharmaceutical preparation of claim 5, wherein component (c) is either dipalmitoylphosphatidylcholine or dimyristoylphosphatidylcholine.

13. The pharmaceutical preparation of claim 5, wherein component (d) comprises nanoparticles.

14. The pharmaceutical preparation of claim 1, wherein the high pressure homogenization is performed at 50 to 1600 bar

15. The pharmaceutical preparation of claim 3, wherein the cytokine is a PEG-cytokine.

16. The pharmaceutical preparation of claim 5, wherein the component (c) is a chemically modified phosphatidylethanolamine to which proteins may be coupled.

17. The pharmaceutical preparation of claim 5, wherein the component (c) is an ether lipid.

18. The pharmaceutical preparation of claim 11, wherein the component (c) is egg phosphatidylcholine.
